(19) United States
(12) Patent Application Publication
(10) Pub. No.: US 2017/0347920 A1
(43) Pub. Date: Dec. 7, 2017

(54) Anthropometric Device For Biometric Measurements

(71) Applicant: Justin Leonard, Scottsdale, AZ (US)

(72) Inventor: Justin Leonard, Scottsdale, AZ (US)

(21) Appl. No.: 15/173,678

(22) Filed: Jun. 5, 2016

(51) Int. Cl. A61B 5/107

(52) U.S. Cl. CPC A61B 5/1072; A61B 2560/0431; A61B 2560/0443

(57) ABSTRACT

Disclosed herein is an anthropometric device. The device includes a base, an upstanding member and a measuring member for measuring one or more anthropometric parameters. The upstanding member has a first end and a second end with the first end secured to the base. The measuring member is suitably connected to the upstanding member. The measuring member has distal ends, the distal ends are extendable relative to first measuring member.

### BACKGROUND OF THE INVENTION

### Field of the Invention

This invention relates to anthropometric devices used for biometric measurements. More particularly, this disclosure relates to a portable device used for measuring the wingspan and other biometric measurements.

### Background

It is well known that biometric measurements may be accurate predictors of athletic achievement. For example, Wingspan measurement is commonly used to predict athletic ability. It is well known that individuals with longer arms tend to have the advantage of executing athletic moves. Longer wingspan makes the athlete play taller than their actual height.

There are a number of sports where a longer wingspan proves beneficial. Among such sports are golf, basketball, football, boxing, volleyball, rowing, martial arts, and swimming to name a few. For example in golf, it is well known that the longer the wingspan relative to body height, the easier it is to develop club head speed during the golf swing. Golfers like Gary Player and other somewhat diminutive golfers generated far more club head speed than others of similar or greater height because of their unusually long wingspan. Long drive champion, Jamie Sadlowski is a mere 5′10″ tall but has twice been the champion over much bigger men. While most attribute his unusual length to his superior wrist strength, it will be noticed by even a casual observer, that his wingspan is extraordinarily long.

Any sport that requires a swinging motion is likewise affected. Thus, hockey, where the stick speed is critically important or football where a quarterback's throwing motion affects the length and speed at which the football is thrown. Simply put in mechanical terms, the longer the arms, the greater the leverage.

Thus, there has long been established a need to accurately measure the wingspan of an athlete or any person involved in physical activity. In fact, quite regularly, a physical examination in a doctor's office includes measurements such as height and weight as well as wingspan.

In a doctor's office, a staff person or two is needed to assist with accurate wingspan measurement. Very often such measurements are done with a tape measure. In such cases, the patient cannot assist because the measurement is done from the subject's middle fingers of each hand. Therefore, it takes two staff, at least, to perform the measurement of the wingspan.

Additionally, when measuring wingspan using a tape measure, there is often times a tendency for the tape to arch over a long, gradual curve. This makes it difficult to secure an accurate reading of the measurement from fingertip to fingertip. In fact, a third assistant is needed to prevent such arching to obtain reliable wingspan measurements.

Another method of measuring wingspan includes securing a tape measure directly to a wall. Similar to the measurements performed in a doctor's office, the subject stands facing away from the wall with his arms extended outwardly. The wingspan measurement is then recorded.

This measuring method can be problematical. The tape measure itself must be perfectly level on the wall for the measurement to be accurate. Next, the subject's arms must be straight, also to insure accuracy. Should the subject's hands or arms touch the wall during the test, then a likely inaccurate measurement will result because the subject's shoulders are retracted too far, which renders a shorter wingspan reading than otherwise.

Discussed above have been methods of obtaining wingspan measurements in a strictly controlled environment. An important use of wingspan measurements takes place on the playing or practice field or in general in open space. There are no walls and finding three assistants to take the wingspan measurement may be difficult. Additionally, the time involved for such above described methods is not practical when there 200 or more players to be measured.

What is needed is a method and structure for simplifying taking wingspan measurements. The time needed for taking such measurements must be shortened to just a few minutes or less. Additionally, the number of assistants needed should not exceed one. Finally, there is an established need to take such measurement on an open field, such as a practice or a playing field.

### SUMMARY OF THE DISCLOSURE

In accordance with the instant disclosure of an anthropometric device is described and claimed which greatly reduces the time required to take biometric measurements. Additionally, the anthropometric device is described and claimed herein can be used on any surface and is easily transported.

The disclosure herein is for an anthropometric device which is capable of measuring more than one biometric parameter. In general, the device includes a base, and of standing member connected to the base, a measurement member connected to the upstanding member. The measurement member has distal ends which are expendable relative to the measurement member. In an exemplary embodiment, the distal ends define finger tabs, which facilitate the ability of a subject to extend the tabs relative to the measurement member.

In accordance with the objects and advantages of the invention that will be appreciated by those skilled in the art upon reading this disclosure and using the apparatus herein, a first exemplary embodiment is disclosed herein which includes:
a base;
an upstanding member having a first end and a second end, the first end secured to the base;
a first measuring member for measuring one or more anthropometric parameters, the first measuring member connected to the upstanding member;
the first measuring member having a distal end, the distal end being extendable relative to first measuring member.

In an exemplary embodiment of the anthropometric device includes first and second measurement members. The first measurement member measuring the biometric parameter of wingspan. The second measurement member measuring the biometric measurement of height.

In another exemplary embodiment of the anthropometric device in accordance with this disclosure, the first and second measurement members define panels. In this embodiment, each of the panels is a removable relative to the upstanding member. In a related exemplary embodiment, each of the panels is rotatable relative to the upstanding member.

### BRIEF DESCRIPTION OF THE DRAWING

For a further understanding of the objects and advantages of the present invention, reference should be had to the following detailed description, taken in conjunction with the accompanying drawing, in which like parts are given like reference numerals and wherein:

FIG. 1 is a perspective view of the anthropometric device in accordance with this disclosure.

FIG. 2 is a perspective view of the anthropometric device with the panels for measuring wingspan in the extended position.

FIG. 3 is an enlarged view of a portion of the anthropometric device of FIG. 3 illustrating the indicia for measuring the biometric value of wingspan.

FIG. 4 is a perspective view of another embodiment of the anthropometric device in accordance with this disclosure with the ability to perform the biometric measurement of height.

FIG. 5 is an enlarged perspective view of the indicia for measuring height of the embodiment of the anthropometric device illustrated in FIG. 4.

FIG. 6 is a perspective view of another embodiment of the anthropometric device in accordance with this disclosure having an ability to rotate a measuring panel relative to the device.

### DETAILED DESCRIPTION OF THE DISCLOSURE

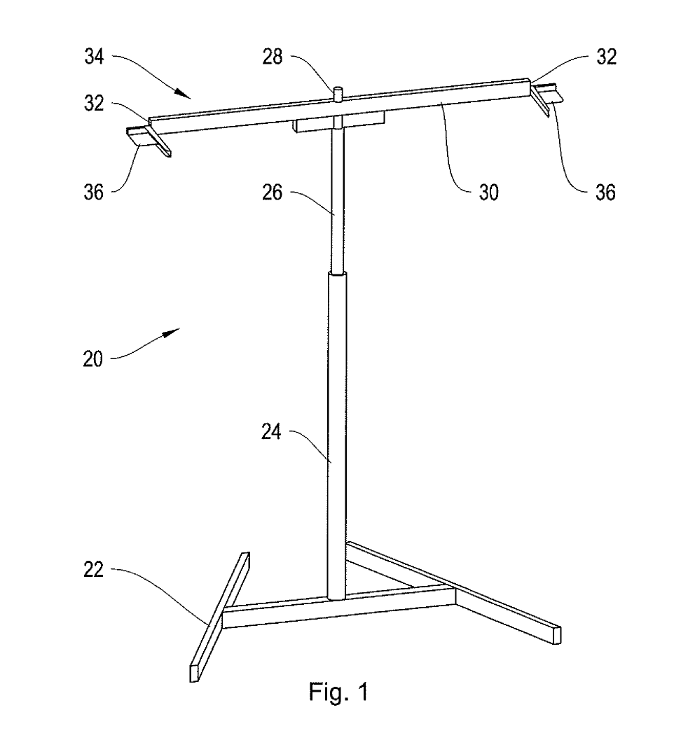

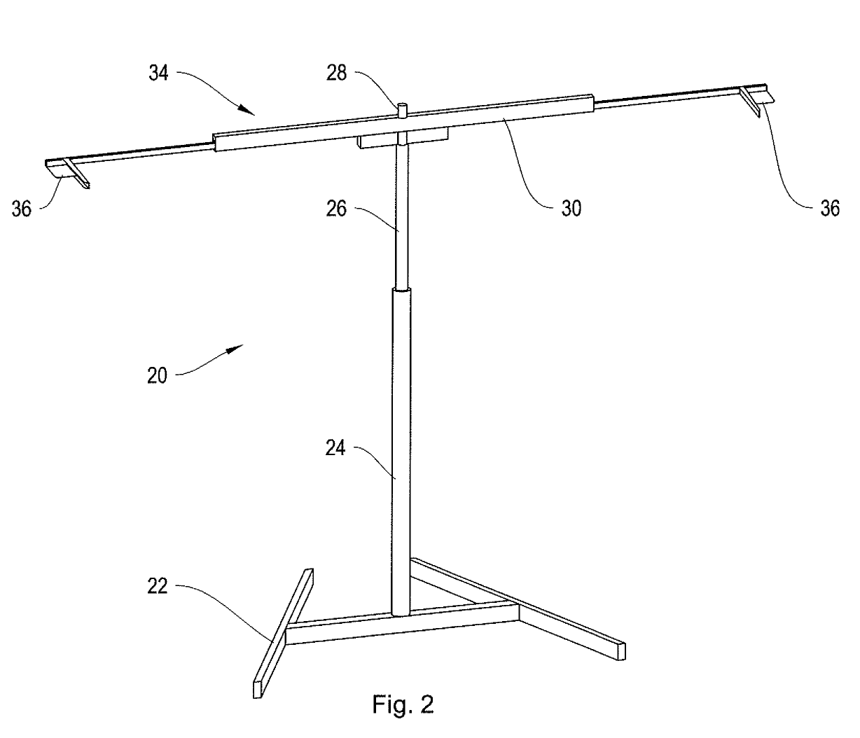

The invention will now be described with respect to FIGS. 1 and 2, which illustrate an exemplary embodiment of the anthropometric device in accordance with this disclosure, shown generally by the numeral 20. The anthropometric device 20 includes a base 22, an upstanding member of 24. In the exemplary embodiment shown in FIG. 1, telescoping members 26 and 28 extended from the upstanding member 24 as illustrated.

A first measuring member defining a first panel 30 is connected to the telescoping member 28 as illustrated in the exemplary embodiment of FIG. 1. The first panel 30 distal ends 32 and a middle zone, generally indicated by the numeral 34. The first panel 30 includes structure for attaching the first panel to this telescoping member 28. In the exemplary embodiment the first panel 30 is threaded with an opening size and shape to adapt to the telescoping member 28. More particularly, the first panel 30 has a midpoint between the distal ends 32 and in the exemplary embodiment of FIG. 1, the first panel 30 is attached to the telescoping member 28 at the midpoint.

As illustrated in FIG. 1, the first panel 30 is designed to measure wingspan. The distal ends 32 include in finger tabs 36 which are expendable from the first panel 30. As is well known in the art, wingspan is defined as the biometric measurement from a person's middle finger of one hand to the middle finger of the other hand. In order to make this measurement, a person would stand with her back toward the upstanding member 24 and using his or her hands extent of the finger tabs 36 with their middle finger as far as possible.

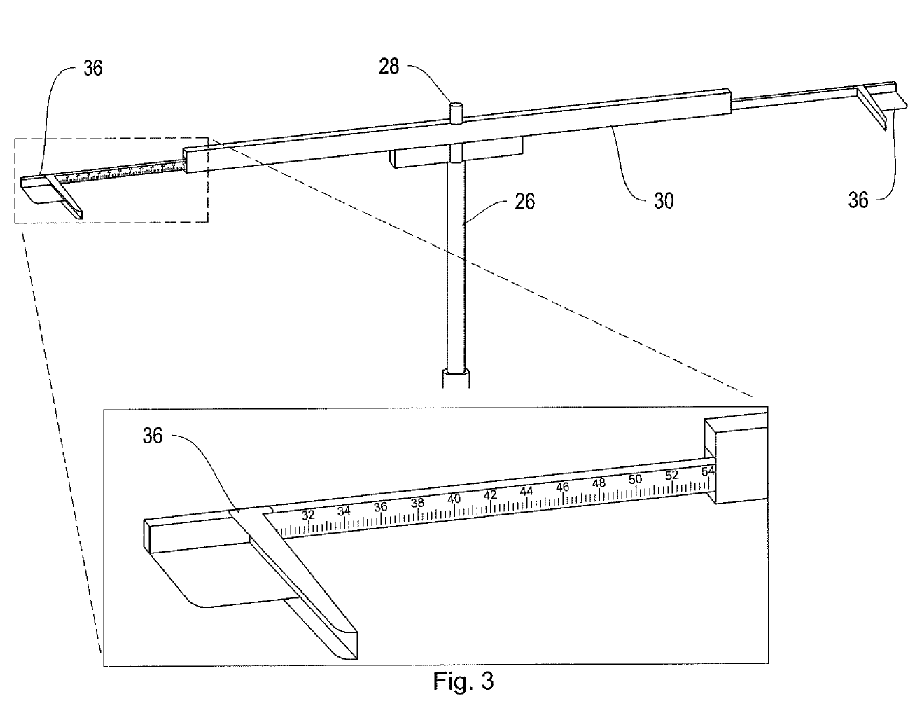

As shown particularly in FIG. 2, the finger tabs 36 are extended. The subject's wingspan is measured by reading the indicia as shown in FIG. 3.

In another exemplary embodiment, the base 22 includes an anchoring member (not shown) for anchoring the case to the desired surface. For example, it is desirable for the base 22 to be used on a grass or dirt playing field. These types of surfaces can be unstable and require additional anchoring methods. Additionally, it is desirable to use the anthropometric device in accordance with this disclosure, on a concrete surface or another hard surface. Mounting members such as concrete bolts are used in those circumstances.

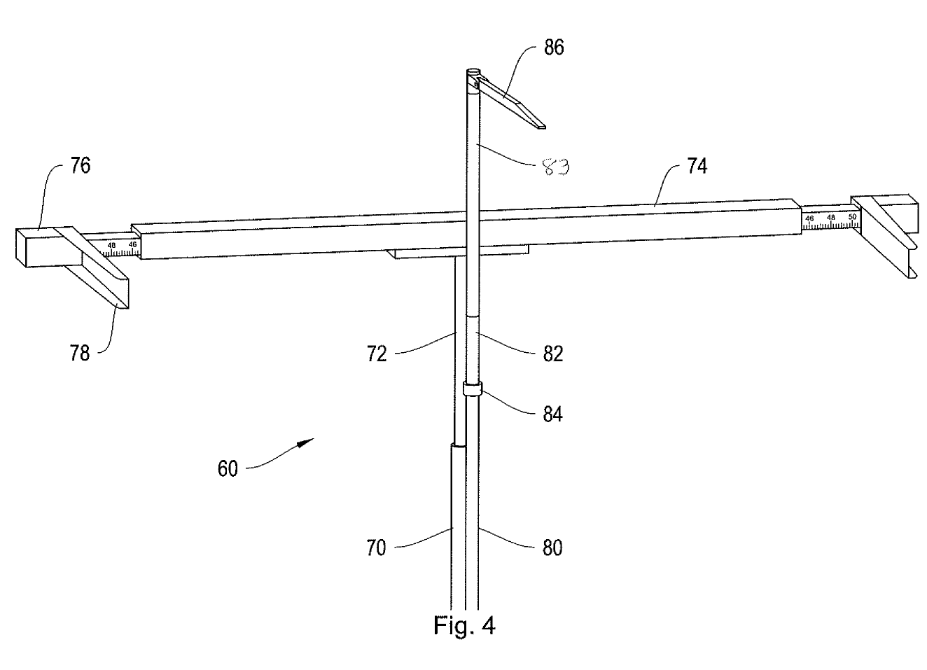

With particular reference to FIG. 4, there is shown an alternative embodiment of the anthropometric device in accordance with this disclosure, generally indicated by the numeral 60. The device 60 is virtually the same as the device 20 described above. However, the device 60 includes the ability to measure the height of a subject.

The device 60 includes an upstanding member 70 having a telescoping member 72. A first measuring member defining a first panel 74 is attached to the telescoping member 72. The first panel 74 has distal ends 76. The distal ends 76 are extendable relative to the first panel 74. And, similar to the exemplary embodiment shown in FIGS. 1-3, the first panel 74 includes finger tabs 78.

Additionally, the device 60 includes a second measuring member defining a second panel 80. The second panel 80 is connected to the upstanding member 70. The second panel 80 includes telescoping members 82 and 83 which are movable within the second panel 80 as is well known in the art. Also, as is well known in the art, a tightening ring 84 is used to lock and unlock the telescoping members in place.

The second panel 80 includes a height bar 86. The subject stands with his or her back to be upstanding member 70 and the second panel is moved so that the height bar 86 placed on top of the subject's head. At that point, the biometric measurement of height can be taken.

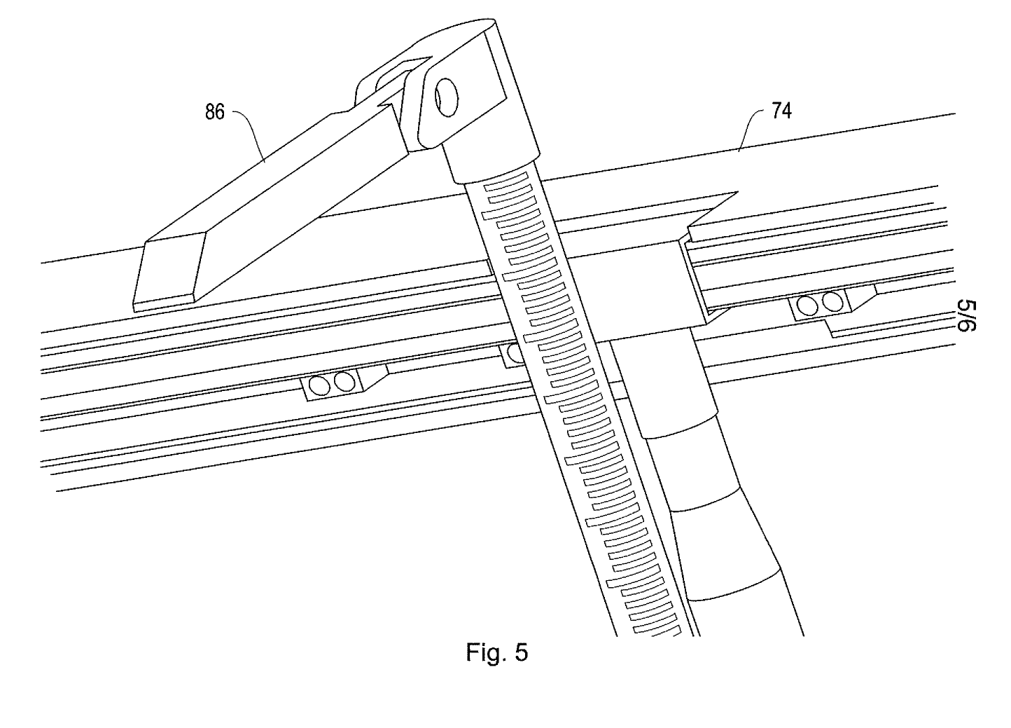

With particular reference to FIG. 5, there is shown another exemplary embodiment of the anthropometric device in accordance with this disclosure. In this exemplary embodiment, the second panel 80 includes indicia adjacent the height bar | 86. The indicia, as illustrated, are easily read and the height of the subject is then easily recorded.

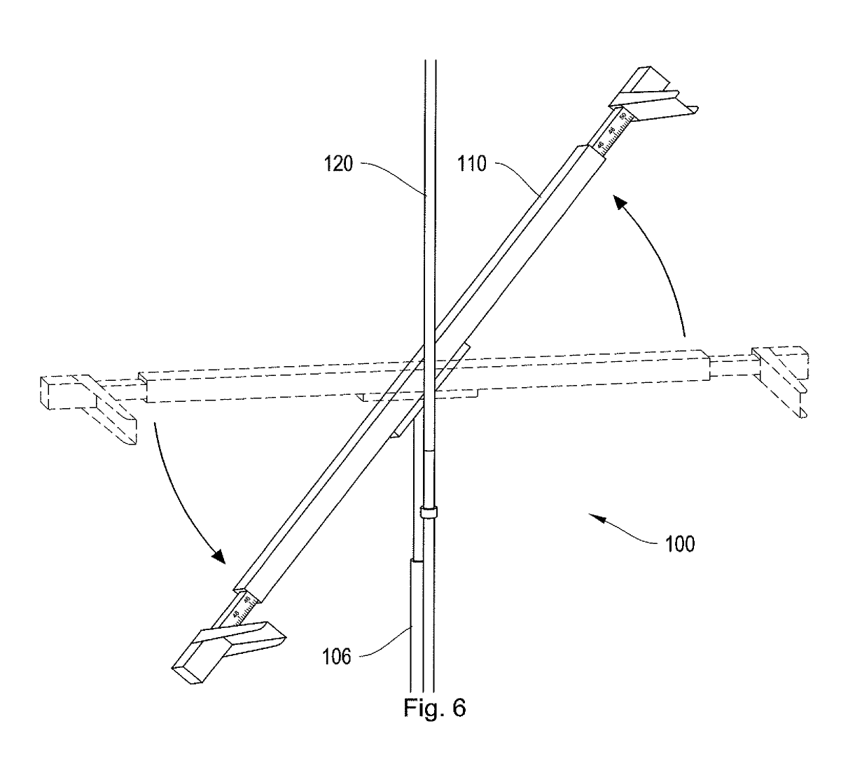

With particular reference to FIG. 6, there is shown another exemplary embodiment of the anthropometric device in accordance with this disclosure based on the exemplary embodiment shown in FIGS. 1-3. In this exemplary embodiment, the device is generally indicated by the numeral 100. The device 100 includes an upstanding member 106 mounted on a base (not shown). The device 100 includes a first panel 110 which is rotatably connected to the upstanding member 106 as illustrated. In this exemplary embodiment, the device one member includes a second panel 120 taking the biometric reading of height.

In another exemplary embodiment, the second panel is similarly attached to the upstanding member in a rotatable fashion. When not in use, the first and second panels are rotated to be coincident with the upstanding member to prevent an accidental collection. Within the spirit and scope of the invention, the first and second panels in another embodiment foldable with respect to the upstanding member. In this exemplary embodiment, the first and second panels are folded when the device is not in use. This again minimizes the chance of accident or collision. In another exemplary embodiment, the first and second panels are removable with respect to the device. This makes the device more easily transportable.

In another exemplary embodiment, the panels are extendable and self-locking. Once the extension needed for biometric measurement is achieved, the panel's lot that that extension. The reading is then recorded and the measurement taken. After the measurement is taken, the self-locking mechanism is released and retracts to its home location.

As will be appreciated by those skilled in the art, electronic measurement, including laser measurement is within the spirit and scope of this invention.

Also as will be appreciated by those skilled in the art, various mechanical structures may be employed to facilitate the ease of extending the panels to the biometric measuring distance. In an exemplary embodiment, a rail of bearing mechanism is contemplated.

In that another exemplary embodiment, the first panel and is rotatably connected to the upstanding member. One end of the first panel includes a height bar. Upon measuring the wingspan of a subject, the first panel is rotated to a plane coincident with the upstanding member so that the height of the subject may then be taken. Thus, one panel measures more than one biometric parameter.

While the foregoing detailed description has described several embodiments of the anthropometric device in accordance with this disclosure, it is to be understood that the above description is illustrative only and not limiting of the anthropometric device. It will be appreciated that the embodiments discussed above and the virtually infinite embodiments that are not mentioned could easily be within the scope and spirit of this invention. Thus, the invention is to be limited only by the claims as set forth below.

What is claimed is:

1. An anthropometric device for making biometric measurements, the device comprising:
a base;
an upstanding member having a first end and a second end, the first end secured to the base;
a first measuring member for measuring one or more anthropometric parameters, the first measuring member connected to the upstanding member;
the first measuring member having a distal end, the distal end being extendable relative to first measuring member.

2. The device as set forth in claim 1, wherein the first measuring member has a first and a second distal end and the first measuring member has a middle zone and the first measuring member is connected to the upstanding member at the middle zone.

3. The device as set forth in claim 2, wherein the first measuring member is connected to the upstanding member at the mid-point of the first measuring member.

4. The device as set forth in claim 2, wherein the first measuring member is connected to the upstanding member at the middle zone and is rotatable with respect to the upstanding member.

5. The device as set forth in claim 3, wherein the first measuring member is connected to the upstanding member at the mid-point and is rotatable with respect to the upstanding member.

6. The device as set forth in claim 1, wherein the first measuring member distal end includes a finger tab for supporting extendable movement by a finger.

7. The device as set forth in claim 1, wherein the base includes an anchor member for anchoring the base to a grass playing field.

8. The device as set forth in claim 1, wherein the base includes an anchor member for anchoring the base to a concrete surface.

9. The device as set forth in claim 1, wherein the device includes a second measuring member for taking a second biometric measurement.

10. The device as set forth in claim 1, wherein the first measuring member is designed for taking the biometric measurement of wingspan.

11. The device as set forth in claim 9, wherein the second measuring member is designed for taking the biometric measurement of height.

12. The device as set forth in claim 11, wherein the second measuring member has a distal end which is moves relative to the second measuring member.

13. The device as set forth in claim 1, wherein the first measuring member includes readable indicia.

14. The device as set forth in claim 9, wherein the second measuring member includes readable indicia.
